(19) United States
(12) Patent Application Publication
(10) Pub. No.: US 2019/0029975 A1
(43) Pub. Date: Jan. 31, 2019

(54) TOCOPHEROL AND TOCOTRIENOL QUINONE DERIVATIVES FOR INCREASING THIOSULFATE LEVELS OR DECREASING HYDROGEN SULFIDE LEVELS

(71) Applicant: BIOELECTRON TECHNOLOGY CORPORATION, Mountain View, CA (US)

(72) Inventor: William D. SHRADER, Belmomt, CA (US)

(21) Appl. No.: 16/069,772

(22) PCT Filed: Jan. 12, 2017
(86) PCT No.: PCT/US2017/013271

§ 371 Date: Jul. 12, 2018

Related U.S. Application Data
(60) Provisional application No. 62/277,486, filed on Jan. 12, 2016.
(60) Provisional application No. 62/277,890, filed on Jan. 12, 2016.
(60) Provisional application No. 62/277,847, filed on Jan. 12, 2016.

(51) Int. Cl. A61K 31/122; A61K 31/05

(52) U.S. Cl. CPC A61K 31/122; G01N 33/502; A61K 31/05

(57) ABSTRACT

The present invention provides, in some embodiments, compounds and compositions that are SQOR substrates, and which are useful in increasing thiosulfate, decreasing hydrogen sulfide, and/or preventing an increase in hydrogen sulfide in a subject, tissue, or cell.

### BACKGROUND

This application claims the benefit under 35 U.S.C. § 119(e) of U.S. Provisional Patent Application No. 62/277,486, TOCOPHEROL AND TOCOTRIENOL QUINONE DERIVATIVES FOR INCREASING THIOSULFATE LEVELS OR DECREASING HYDROGEN SULFIDE LEVELS, filed Jan. 12, 2016, also U.S. Provisional Patent Application No. 62/277,847, titled METHODS FOR DIAGNOSING AND TREATING OXIDATIVE STRESS DISORDERS USING BIOMARKERS, filed Jan. 12, 2016, and also U.S. Provisional Patent Application No. 62/277,890, titled TOCOPHEROL AND TOCOTRIENOL QUINONE DERIVATIVES FOR INCREASING THIOSULFATE LEVELS OR DECREASING HYDROGEN SULFIDE LEVELS, filed Jan. 12, 2016, the disclosures of each of which are incorporated by reference herein in their entirety.

Hydrogen sulfide (H₂S) has been identified as a biological signaling molecule with increasingly studied functions. To date, hydrogen sulfide has been postulated to function as a neuromodulator, as a neuroprotectant, as a protectant from ischemia and reperfusion injury, as an oxygen sensor, as a mediator of vasodilation, as a promoter of angiogenesis, and as a modulator of inflammation. See Jackson, et al., 2012, Biochemistry 51:6804-6815. Metabolism of hydrogen sulfide is postulated to initiate its signaling effects. This metabolism is initiated in the mitochondria by the enzyme sulfide: quinone oxidoreductase (EC 1.8.5.4) in prokaryotes and eukaryotes. Shahak, et al., 2008, Advances in Photosynthesis and Respiration, 320-335; and Hell, et al., eds., Springer, Heidelberg, Germany. To date, sulfide:quinone oxidoreductases have been expressed from both prokaryotic and eukaryotic sources.

Elevated hydrogen sulfide levels in mammals, whether resulting from exogeneous sources or from internal normal or abnormal biological processes, result in symptoms ranging from lethargy, decreased heart and respiration rate, hibernation, neurological symptoms mimicking those found in Leigh's Syndrome, and death.

Sulfide:quinone oxidoreductase (SQOR) converts hydrogen sulfide into thiosulfate in vivo.

Few if any modulators of the key enzyme sulfide:quinone oxidoreductase are known. Compounds capable of modulating sulfide: quinone oxidoreductase activity would be useful for modulating signaling and other properties of hydrogen sulfide and thiosulfate in cells and organisms.

### DESCRIPTION OF THE FIGURES

FIG. 1 is a plot of [thiosulfate] concentration as a function of EPI-743 administration time, as described in Example 3.

FIG. 2 is a graph of Intracellular [cysteine] for EPI-743 Treated ATG-Stressed Cells, as described in Example 4.

FIG. 3 is a graph of Intracellular [thiosulfate] for EPI-743 Treated ATG-Stressed Cells, as described in Example 4.

### SUMMARY OF THE INVENTION

The present invention provides, in some embodiments, compounds and compositions that are SQOR substrates, and which are useful for increasing thiosulfate, decreasing hydrogen sulfide, and/or preventing an increase in hydrogen sulfide in a subject, tissue, or cell. The invention further provides methods for using such compounds and compositions.

The present invention provides, in some embodiments, compounds and compositions that are SQOR substrates, and which are useful for increasing thiosulfate, and/or decreasing hydrogen sulfide in a subject, tissue, or cell. The invention further provides methods for using such compounds and compositions. In some examples, the subject is a human. The present invention provides, in some embodiments, methods of screening for identifying molecules capable of modulating the activity of a sulfide:quinone oxidoreductase. In some examples, provided herein are sulfide:quinone oxidoreductase which can be any sulfide:quinone oxidoreductase known to those of skill, including any sulfide:quinone oxidoreductase provided herein. In some examples, the methods include the steps of contacting a sulfide:quinone oxidoreductase or a cell expressing a sulfide:quinone oxidoreductase with a test molecule and measuring at least one activity of the sulfide: quinone oxidoreductase. In some examples, the molecules that modulate sulfide:quinone oxidoreductase activity are identified as molecules capable of modulating its activity. In some examples, these molecules are provided herein. Molecules identified by the screening assays can be administered to a subject for any method of treatment or prevention described herein. Exemplary test molecules include small organic molecules (e.g., with a molecular weight less than 1 kD), that may be obtained by organic synthesis or combinatorial chemistry; nucleic acids and proteins. In some examples, these molecules are provided herein.

In one aspect of the invention is a method of increasing a thiosulfate level, decreasing a hydrogen sulfide level, or preventing an increased hydrogen sulfide level in a subject, comprising: administering to the subject an effective amount of an agent selected from the group consisting of: compounds of Formula A, Formula I, Formula I-Unsat, Formula I-Sat, Formula II, Formula II-Unsat, Formula II-Sat, Formula III, Formula III-Unsat, Formula III-Sat, Formula IV, Formula IV-Unsat, Formula IV-Unsat-R, Formula IV-Unsat-S, Formula IV-Sat, Formula IV-Sat-R, Formula IV-Sat-S, Formula V, Formula V-Unsat, Formula V-Sat, Formula VI, Formula VI-Unsat, Formula VI-Unsat-R, Formula VI-Unsat-S, Formula VI-Sat, Formula VI-Sat-R, Formula VI-Sat-S, a hydroquinone of Formula A, a hydroquinone of Formula I, a hydroquinone of Formula I-Unsat, a hydroquinone of Formula I-Sat, a hydroquinone of Formula II, a hydroquinone of Formula II-Unsat, a hydroquinone of Formula II-Sat, a hydroquinone of Formula III, a hydroquinone of Formula III-Unsat, a hydroquinone of Formula III-Sat, a hydroquinone of Formula IV, a hydroquinone of Formula IV-Unsat, a hydroquinone of Formula IV-Unsat-R, a hydroquinone of Formula IV-Unsat-S, a hydroquinone of Formula IV-Sat, a hydroquinone of Formula IV-Sat-R, a hydroquinone of Formula IV-Sat-S, a hydroquinone of Formula V, a hydroquinone of Formula V-Unsat, a hydroquinone of Formula V-Sat, a hydroquinone of Formula VI, a hydroquinone of Formula VI-Unsat, a hydroquinone of Formula VI-Unsat-R, a hydroquinone of Formula VI-Unsat-S, a hydroquinone of Formula VI-Sat, a hydroquinone of Formula VI-Sat-R, a hydroquinone of Formula VI-Sat-S, alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof. In some embodiments, the agent is selected from the group consisting of compounds of Formula IV:

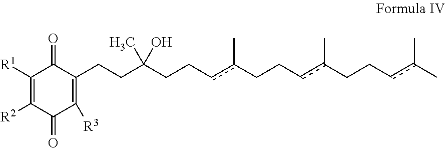

wherein each bond indicated with a dashed line, independently of the other bonds indicated with a dashed line, can be a single bond or a double bond; and R¹, R², and R³ are independently selected from the group consisting of H, (C₁-C₄)-alkyl, (C₁-C₄)-haloalkyl, —CN, —F, —Cl, —Br, and —I; and a stereoisomer, mixtures of stereoisomers, salt, hydrate, or solvate thereof; or the hydroquinone form thereof. In some embodiments, the agent is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof. In some embodiments, the agent is selected from the group consisting of alpha-tocotrienol quinone, beta-tocotrienol quinone, gamma-tocotrienol quinone, and delta-tocotrienol quinone, and salts, stereoisomers, mixtures of stereoisomers, hydrates, and solvates thereof. In some embodiments, the agent is selected from the group consisting of alpha-tocotrienol hydroquinone, beta-tocotrienol hydroquinone, gamma-tocotrienol hydroquinone, and delta-tocotrienol hydroquinone, and salts, stereoisomers, mixtures of stereoisomers, hydrates, and solvates thereof. In some embodiments, the agent is selected from the group consisting of alpha-tocotrienol quinone, alpha-tocotrienol hydroquinone, and salts, stereoisomers, mixtures of stereoisomers, hydrates, and solvates thereof. In some embodiments, the agent is selected from the group consisting of alpha-tocotrienol quinone, and stereoisomers, mixtures of stereoisomers, hydrates, and solvates thereof. In some embodiments, the agent is selected from the group consisting of alpha-tocotrienol hydroquinone, and stereoisomers, mixtures of stereoisomers, hydrates, and solvates thereof. In some embodiments, the agent is:

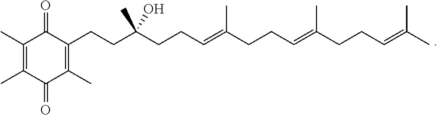 [SMILES: CC(C)=CCC/C(C)=C/CC/C(C)=C/CC[C@@](C)(O)CCC1=C(C)C(=O)C(C)=C(C)C1=O]

In some embodiments, the agent is:

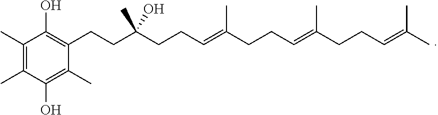 [SMILES: CC(C)=CCC/C(C)=C/CC/C(C)=C/CC[C@@](C)(O)CCc1c(C)c(O)c(C)c(C)c1O]

In some embodiments, including any of the foregoing embodiments, the agent is administered in a composition comprising the agent and a pharmaceutically acceptable carrier. In some embodiments, including any of the foregoing embodiments, the method comprises increasing a thiosulfate level in the subject. In some embodiments, including any of the foregoing embodiments, the method is selected from the group consisting of: antidoting cyanide poisoning, preserving renal function, treatment of acute lung injury, and treatment or prevention of calciphylaxis in blood vessels, kidney toxicity in cancer therapy, antibacterial infection, anti-fungal infection, ulcerative colitis, hypertension, and proteinuria. In some embodiments, including any of the foregoing embodiments, the method comprises decreasing a hydrogen sulfide level in the subject. In some embodiments, including any of the foregoing embodiments, the method comprises preventing an increased hydrogen sulfide level in the subject. In some embodiments, including any of the foregoing embodiments, the subject has been exposed to exogenous hydrogen sulfide. In some embodiments, including any of the foregoing embodiments, the subject may be exposed to exogenous hydrogen sulfide. In some embodiments, including any of the foregoing embodiments, the subject suffers from a disorder that results in an increased H₂S level. In some embodiments, including any of the foregoing embodiments, the disorder is not a mitochondrial disorder. In some embodiments, including any of the foregoing embodiments, the disorder is not Leigh Syndrome. In some embodiments, including any of the foregoing embodiments, the subject does not suffer from a disorder that results in an increased H₂S level. In some embodiments, including any of the foregoing embodiments, the method comprises increasing respiration rate in the subject. In some embodiments, including any of the foregoing embodiments, the agent is administered orally. In some embodiments, including any of the foregoing embodiments, the agent is administered by injection. In some embodiments, including any of the foregoing embodiments, the agent is administered topically. In some embodiments, including any of the foregoing embodiments, the agent is administered in single or multiple doses in an amount independently selected from about 0.1 mg/kg body weight to about 300 mg/kg body weight. In some embodiments, including any of the foregoing embodiments, the agent is administered in single or multiple doses in an amount independently selected from about 1 mg/kg body weight to about 20 mg/kg body weight. In some embodiments, including any of the foregoing embodiments, the agent is administered in single or multiple doses in an amount independently selected from about 5 mg/kg body weight to about 15 mg/kg body weight. In some embodiments, including any of the foregoing embodiments, the thiosulfate or hydrogen sulfide concentration is measured in whole blood, plasma, serum, white blood cells, red blood cells, or cerebrospinal fluid. In some embodiments, including any of the foregoing embodiments, the thiosulfate or hydrogen sulfide concentration is measured in leukocytes. In some embodiments, including any of the foregoing embodiments, the thiosulfate or hydrogen sulfide concentration is measured in plasma.

In another aspect of the invention is a method of inducing and then reversing a temporarily decreased metabolic state in a subject or tissue, comprising: (1) administering hydrogen sulfide to the subject or the tissue to decrease the metabolic state of the subject or tissue, and (2) subsequently administering to the subject or the tissue an effective amount of an agent selected from the group consisting of: compounds of Formula A, Formula I, Formula I-Unsat, Formula I-Sat, Formula II, Formula II-Unsat, Formula II-Sat, Formula III, Formula III-Unsat, Formula III-Sat, Formula IV, Formula IV-Unsat, Formula IV-Unsat-R, Formula IV-Unsat-S, Formula IV-Sat, Formula IV-Sat-R, Formula IV-Sat-S, Formula V, Formula V-Unsat, Formula V-Sat, Formula VI, Formula VI-Unsat, Formula VI-Unsat-R, Formula VI-Unsat-S, Formula VI-Sat, Formula VI-Sat-R, Formula VI-Sat-S, a hydroquinone of Formula A, a hydroquinone of Formula I, a hydroquinone of Formula I-Unsat, a hydroquinone of Formula I-Sat, a hydroquinone of Formula II, a hydroquinone of Formula II-Unsat, a hydroquinone of Formula II-Sat, a hydroquinone of Formula III, a hydroquinone of Formula III-Unsat, a hydroquinone of Formula III-Sat, a hydroquinone of Formula IV, a hydroquinone of Formula IV-Unsat, a hydroquinone of Formula IV-Unsat-R, a hydroquinone of Formula IV-Unsat-S, a hydroquinone of Formula IV-Sat, a hydroquinone of Formula IV-Sat-R, a hydroquinone of Formula IV-Sat-S, a hydroquinone of Formula V, a hydroquinone of Formula V-Unsat, a hydroquinone of Formula V-Sat, a hydroquinone of Formula VI, a hydroquinone of Formula VI-Unsat, a hydroquinone of Formula VI-Unsat-R, a hydroquinone of Formula VI-Unsat-S, a hydroquinone of Formula VI-Sat, a hydroquinone of Formula VI-Sat-R, a hydroquinone of Formula VI-Sat-S, alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof. In some embodiments, the agent is selected from the group consisting of compounds of Formula IV:

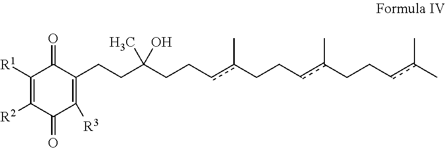

wherein each bond indicated with a dashed line, independently of the other bonds indicated with a dashed line, can be a single bond or a double bond; and R¹, R², and R³ are independently selected from the group consisting of H, (C₁-C₄)-alkyl, (C₁-C₄)-haloalkyl, —CN, —F, —Cl, —Br, and —I; and a stereoisomer, mixtures of stereoisomers, salt, hydrate, or solvate thereof; or the hydroquinone form thereof. In some embodiments, the agent is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof. In some embodiments, the agent is selected from the group consisting of alpha-tocotrienol quinone, beta-tocotrienol quinone, gamma-tocotrienol quinone, and delta-tocotrienol quinone, and salts, stereoisomers, mixtures of stereoisomers, hydrates, and solvates thereof. In some embodiments, the agent is selected from the group consisting of alpha-tocotrienol hydroquinone, beta-tocotrienol hydroquinone, gamma-tocotrienol hydroquinone, and delta-tocotrienol hydroquinone, and salts, stereoisomers, mixtures of stereoisomers, hydrates, and solvates thereof. In some embodiments, the agent is selected from the group consisting of alpha-tocotrienol quinone, alpha-tocotrienol hydroquinone, and salts, stereoisomers, mixtures of stereoisomers, hydrates, and solvates thereof. In some embodiments, the agent is selected from the group consisting of alpha-tocotrienol quinone, and stereoisomers, mixtures of stereoisomers, hydrates, and solvates thereof. In some embodiments, the agent is selected from the group consisting of alpha-tocotrienol hydroquinone, and stereoisomers, mixtures of stereoisomers, hydrates, and solvates thereof. In some embodiments, the agent is:

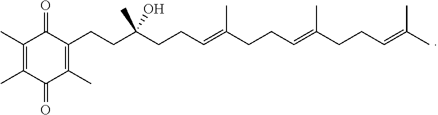 [SMILES: CC(C)=CCC/C(C)=C/CC/C(C)=C/CC[C@@](C)(O)CCC1=C(C)C(=O)C(C)=C(C)C1=O]

In some embodiments, the agent is:

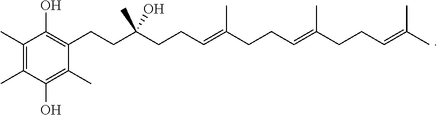 [SMILES: CC(C)=CCC/C(C)=C/CC/C(C)=C/CC[C@@](C)(O)CCc1c(C)c(O)c(C)c(C)c1O]

In some embodiments, including any of the foregoing embodiments, the agent is administered in a composition comprising the agent and a pharmaceutically acceptable carrier. In some embodiments, including any of the foregoing embodiments, the agent is administered orally. In some embodiments, including any of the foregoing embodiments, the agent is administered by injection. In some embodiments, including any of the foregoing embodiments, the agent is administered topically. In some embodiments, including any of the foregoing embodiments, the agent is administered in single or multiple doses in an amount independently selected from about 0.1 mg/kg body weight to about 300 mg/kg body weight. In some embodiments, including any of the foregoing embodiments, the agent is administered in single or multiple doses in an amount independently selected from about 1 mg/kg body weight to about 20 mg/kg body weight. In some embodiments, including any of the foregoing embodiments, the agent is administered in single or multiple doses in an amount independently selected from about 5 mg/kg body weight to about 15 mg/kg body weight. In some embodiments, including any of the foregoing embodiments, the thiosulfate or hydrogen sulfide concentration is measured in whole blood, plasma, serum, white blood cells, red blood cells, or cerebrospinal fluid. In some embodiments, including any of the foregoing embodiments, the thiosulfate or hydrogen sulfide concentration is measured in leukocytes. In some embodiments, including any of the foregoing embodiments, the thiosulfate or hydrogen sulfide concentration is measured in plasma.

In another aspect of the invention is a method of reversing a temporarily decreased metabolic state in a subject or tissue, comprising: (1) providing a subject or tissue with a decreased metabolic state, and (2) subsequently administering to the subject or the tissue an effective amount of an agent selected from the group consisting of: compounds of Formula A, Formula I, Formula I-Unsat, Formula I-Sat, Formula II, Formula II-Unsat, Formula II-Sat, Formula III, Formula III-Unsat, Formula III-Sat, Formula IV, Formula IV-Unsat, Formula IV-Unsat-R, Formula IV-Unsat-S, Formula IV-Sat, Formula IV-Sat-R, Formula IV-Sat-S, Formula V, Formula V-Unsat, Formula V-Sat, Formula VI, Formula VI-Unsat, Formula VI-Unsat-R, Formula VI-Unsat-S, Formula VI-Sat, Formula VI-Sat-R, Formula VI-Sat-S, a hydroquinone of Formula A, a hydroquinone of Formula I, a hydroquinone of Formula I-Unsat, a hydroquinone of Formula I-Sat, a hydroquinone of Formula II, a hydroquinone of Formula II-Unsat, a hydroquinone of Formula II-Sat, a hydroquinone of Formula III, a hydroquinone of Formula III-Unsat, a hydroquinone of Formula III-Sat, a hydroquinone of Formula IV, a hydroquinone of Formula IV-Unsat, a hydroquinone of Formula IV-Unsat-R, a hydroquinone of Formula IV-Unsat-S, a hydroquinone of Formula IV-Sat, a hydroquinone of Formula IV-Sat-R, a hydroquinone of Formula IV-Sat-S, a hydroquinone of Formula V, a hydroquinone of Formula V-Unsat, a hydroquinone of Formula V-Sat, a hydroquinone of Formula VI, a hydroquinone of Formula VI-Unsat, a hydroquinone of Formula VI-Unsat-R, a hydroquinone of Formula VI-Unsat-S, a hydroquinone of Formula VI-Sat, a hydroquinone of Formula VI-Sat-R, a hydroquinone of Formula VI-Sat-S, alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof. In some embodiments, the agent is selected from the group consisting of compounds of Formula IV:

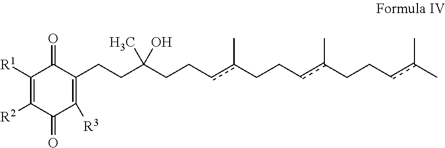

wherein each bond indicated with a dashed line, independently of the other bonds indicated with a dashed line, can be a single bond or a double bond; and R¹, R², and R³ are independently selected from the group consisting of H, (C₁-C₄)-alkyl, (C₁-C₄)-haloalkyl, —CN, —F, —Cl, —Br, and —I; and a stereoisomer, mixtures of stereoisomers, salt, hydrate, or solvate thereof; or the hydroquinone form thereof. In some embodiments, the agent is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof. In some embodiments, the agent is selected from the group consisting of alpha-tocotrienol quinone, beta-tocotrienol quinone, gamma-tocotrienol quinone, and delta-tocotrienol quinone, and salts, stereoisomers, mixtures of stereoisomers, hydrates, and solvates thereof. In some embodiments, the agent is selected from the group consisting of alpha-tocotrienol hydroquinone, beta-tocotrienol hydroquinone, gamma-tocotrienol hydroquinone, and delta-tocotrienol hydroquinone, and salts, stereoisomers, mixtures of stereoisomers, hydrates, and solvates thereof. In some embodiments, the agent is selected from the group consisting of alpha-tocotrienol quinone, alpha-tocotrienol hydroquinone, and salts, stereoisomers, mixtures of stereoisomers, hydrates, and solvates thereof. In some embodiments, the agent is selected from the group consisting of alpha-tocotrienol quinone, and stereoisomers, mixtures of stereoisomers, hydrates, and solvates thereof. In some embodiments, the agent is selected from the group consisting of alpha-tocotrienol hydroquinone, and stereoisomers, mixtures of stereoisomers, hydrates, and solvates thereof. In some embodiments, the agent is:

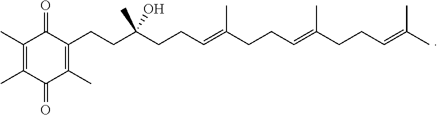 [SMILES: CC(C)=CCC/C(C)=C/CC/C(C)=C/CC[C@@](C)(O)CCC1=C(C)C(=O)C(C)=C(C)C1=O]

In some embodiments, the agent is:

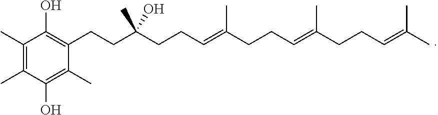 [SMILES: CC(C)=CCC/C(C)=C/CC/C(C)=C/CC[C@@](C)(O)CCc1c(C)c(O)c(C)c(C)c1O]

In some embodiments, including any of the foregoing embodiments, the agent is administered in a composition comprising the agent and a pharmaceutically acceptable carrier. In some embodiments, including any of the foregoing embodiments, the agent is administered orally. In some embodiments, including any of the foregoing embodiments, the agent is administered by injection. In some embodiments, including any of the foregoing embodiments, the agent is administered topically. In some embodiments, including any of the foregoing embodiments, the agent is administered in single or multiple doses in an amount independently selected from about 0.1 mg/kg body weight to about 300 mg/kg body weight. In some embodiments, including any of the foregoing embodiments, the agent is administered in single or multiple doses in an amount independently selected from about 1 mg/kg body weight to about 20 mg/kg body weight. In some embodiments, including any of the foregoing embodiments, the agent is administered in single or multiple doses in an amount independently selected from about 5 mg/kg body weight to about 15 mg/kg body weight. In some embodiments, including any of the foregoing embodiments, the thiosulfate or hydrogen sulfide concentration is measured in whole blood, plasma, serum, white blood cells, red blood cells, or cerebrospinal fluid. In some embodiments, including any of the foregoing embodiments, the thiosulfate or hydrogen sulfide concentration is measured in leukocytes. In some embodiments, including any of the foregoing embodiments, the thiosulfate or hydrogen sulfide concentration is measured in plasma. In some examples, the subject or tissue with a decreased metabolic state is provided by administering hydrogen sulfide to the subject or tissue to decrease the metabolic state of the subject or tissue. In some or any embodiments disclosed herein the compound, composition, or agent is not a prodrug, salt, phosphate substituted form, crystalline form, non-crystalline form, isotopologue, deuterated form, hydrate, or solvate thereof. In some or any embodiments disclosed herein the compound, composition, or agent is a salt, a stereoisomer, a mixture of stereoisomers, a hydrate, or solvate thereof. In some or any embodiments disclosed herein the compound, composition, or agent is a stereoisomer or a mixture of stereoisomers thereof.

In another embodiment of the invention, including any of the foregoing embodiments, the agent is selected from one or more compounds of Formula A, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula A, and all stereoisomers, mixtures of stereoisomers, phosphate substituted forms, and deuterated forms, thereof; or the hydroquinones form thereof. In another embodiment of the invention, including any of the foregoing embodiments, the agent is selected from one or more compounds of Formula A, and all stereoisomers, and mixtures of stereoisomers thereof; or the hydroquinones form thereof.

In another embodiment of the invention, including any of the foregoing embodiments, the agent is selected from one or more compounds of Formula I, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula I, and all stereoisomers, mixtures of stereoisomers, phosphate substituted forms, and deuterated forms, thereof; or the hydroquinones form thereof. In another embodiment of the invention, including any of the foregoing embodiments, the agent is selected from one or more compounds of Formula I, and all stereoisomers, and mixtures of stereoisomers thereof; or the hydroquinones form thereof.

In another embodiment of the invention, including any of the foregoing embodiments, the agent is selected from one or more compounds of Formula I-Unsat, and all stereoisomers, mixtures of stereoisomers, salts, phosphate substituted forms, crystalline forms, non-crystalline forms, deuterated forms, hydrates and solvates thereof; or the hydroquinones form thereof. In another embodiment of the invention, including any of the foregoing embodiments, the agent is selected from one or more compounds of Formula I-Unsat, and all stereoisomers, mixtures of stereoisomers, phosphate substituted forms, and deuterated forms, thereof; or the hydroquinones form thereof. In another embodiment of the invention, including any of the foregoing embodiments, the agent is selected from one or more compounds of Formula I-Unsat, and all stereoisomers, and mixtures of stereoisomers thereof; or the hydroquinone forms thereof.

In another embodiment of the invention, including any of the foregoing embodiments, the agent is selected from one or more compounds of Formula I-Sat, and all stereoisomers, mixtures of stereoisomers, salts, phosphate substituted forms, crystalline forms, non-crystalline forms, deuterated forms, hydrates and solvates thereof; or the hydroquinones form thereof. In another embodiment of the invention, including any of the foregoing embodiments, the agent is selected from one or more compounds of Formula I-Sat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula I-Sat, and all stereoisomers, and mixtures of stereoisomers thereof; or the hydroquinone forms thereof.

In another embodiment of the invention, including any of the foregoing embodiments, the agent is selected from one or more compounds of Formula II, and all stereoisomers, mixtures of stereoisomers, salts, phosphate substituted forms, crystalline forms, non-crystalline forms, deuterated forms, hydrates and solvates thereof; or the hydroquinones form thereof. In another embodiment of the invention, including any of the foregoing embodiments, the agent is selected from one or more compounds of Formula II, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula II, and all stereoisomers, and mixtures of stereoisomers thereof; or the hydroquinone forms thereof.

In another embodiment of the invention, including any of the foregoing embodiments, the agent is selected from one or more compounds of Formula II-Unsat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula II-Unsat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula II-Unsat, and all stereoisomers, and mixtures of stereoisomers thereof; or the hydroquinone forms thereof.

In another embodiment of the invention, including any of the foregoing embodiments, the agent is selected from one or more compounds of Formula II-Sat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula II-Sat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula II-Sat, and all stereoisomers, and mixtures of stereoisomers thereof; or the hydroquinone forms thereof.

In another embodiment of the invention, including any of the foregoing embodiments, the agent is selected from one or more compounds of Formula III, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula III, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula III, and all stereoisomers, and mixtures of stereoisomers thereof; or the hydroquinone forms thereof.

In another embodiment of the invention, including any of the foregoing embodiments, the agent is selected from one or more compounds of Formula III-Unsat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula III-Unsat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula III-Unsat, and all stereoisomers, and mixtures of stereoisomers thereof; or the hydroquinone forms thereof.

In another embodiment of the invention, including any of the foregoing embodiments, the agent is selected from one or more compounds of Formula III-Sat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula III-Sat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula III-Sat, and all stereoisomers, and mixtures of stereoisomers thereof; or the hydroquinone forms thereof.

In another embodiment of the invention, including any of the foregoing embodiments, the agent is selected from one or more compounds of Formula IV, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula IV, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula IV, and all stereoisomers, and mixtures of stereoisomers thereof; or the hydroquinone forms thereof.

In another embodiment of the invention, including any of the foregoing embodiments, the agent is selected from one or more compounds of Formula IV-Unsat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula IV-Unsat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula IV-Unsat, and all stereoisomers, and mixtures of stereoisomers thereof; or the hydroquinone forms thereof.

In another embodiment of the invention, including any of the foregoing embodiments, the agent is selected from one or more compounds of Formula IV-Sat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula IV-Sat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula IV-Sat, and all stereoisomers, and mixtures of stereoisomers thereof; or the hydroquinone forms thereof.

In another embodiment of the invention, including any of the foregoing embodiments, the agent is selected from one or more compounds of Formula V, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula V, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula V, and all stereoisomers, and mixtures of stereoisomers thereof; or the hydroquinone forms thereof.

In another embodiment of the invention, including any of the foregoing embodiments, the agent is selected from one or more compounds of Formula V-Unsat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula V-Unsat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula V-Unsat, and all stereoisomers, and mixtures of stereoisomers thereof; or the hydroquinone forms thereof.

In another embodiment of the invention, including any of the foregoing embodiments, the agent is selected from one or more compounds of Formula V-Sat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula V-Sat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula V-Sat, and all stereoisomers, and mixtures of stereoisomers thereof; or the hydroquinone forms thereof.

In another embodiment of the invention, including any of the foregoing embodiments, the agent is selected from one or more compounds of Formula VI, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula VI, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula VI, and all stereoisomers, and mixtures of stereoisomers thereof; or the hydroquinone forms thereof.

In another embodiment of the invention, including any of the foregoing embodiments, the agent is selected from one or more compounds of Formula VI-Unsat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula VI-Unsat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula VI-Unsat, and all stereoisomers, and mixtures of stereoisomers thereof; or the hydroquinone forms thereof.

In another embodiment of the invention, including any of the foregoing embodiments, the agent is selected from one or more compounds of Formula VI-Sat, and all stereoisomers, mixtures of stereoisomers, salts, phosphate substituted forms, crystalline forms, non-crystalline forms, deuterated forms, hydrates and solvates thereof; or the hydroquinone forms thereof. In another embodiment of the invention, including any of the foregoing embodiments, the agent is selected from one or more compounds of Formula VI-Sat, and all stereoisomers, mixtures of stereoisomers, phosphate substituted forms, and deuterated forms, thereof; or the hydroquinone forms thereof. In another embodiment of the invention, including any of the foregoing embodiments, the agent is selected from one or more compounds of Formula VI-Sat, and all stereoisomers, and mixtures of stereoisomers thereof; or the hydroquinone forms thereof.

In another embodiment of the invention, including any of the foregoing embodiments, the agent comprises one or more compounds selected from the group consisting of alpha-tocotrienol quinone, beta-tocotrienol quinone, gamma-tocotrienol quinone, and delta-tocotrienol quinone. In one embodiment, the agent comprises alpha-tocotrienol quinone. In one embodiment, the agent comprises beta-tocotrienol quinone. In one embodiment, the agent comprises gamma-tocotrienol quinone. In one embodiment, the agent comprises delta-tocotrienol quinone. In some or any embodiments, the alpha, beta, gamma, and delta-tocotrienol quinones have the naturally-occurring stereochemistry, i.e. 3R-hydroxy-6E-10E.

In another embodiment of the invention, including any of the foregoing embodiments, the agent comprises one or more compounds selected from the group consisting of alpha-tocotrienol hydroquinone, beta-tocotrienol hydroquinone, gamma-tocotrienol hydroquinone, and delta-tocotrienol hydroquinone. In one embodiment, the agent comprises alpha-tocotrienol hydroquinone. In one embodiment, the agent comprises beta-tocotrienol hydroquinone. In one embodiment, the agent comprises gamma-tocotrienol hydroquinone. In one embodiment, the agent comprises delta-tocotrienol hydroquinone. In some or any embodiments, the alpha, beta, gamma, and delta-tocotrienol hydroquinones have the naturally-occurring stereochemistry, i.e. 3R-hydroxy-6E-10E.

In another embodiment of the invention, including any of the foregoing embodiments, the agent comprises one or more compounds selected from the group consisting of alpha-tocopherol quinone, beta-tocopherol quinone, gamma-tocopherol quinone, and delta-tocopherol quinone. In one embodiment, the agent comprises alpha-tocopherol quinone. In one embodiment, the agent comprises beta-tocopherol quinone. In one embodiment, the agent comprises gamma-tocopherol quinone. In one embodiment, the agent comprises delta-tocopherol quinone. In some or any embodiments, the alpha, beta, gamma, and delta-tocopherol quinones have the naturally-occurring stereochemistry, i.e. 3(R)-hydroxy and 7(R)-methyl and 11(R)-methyl on the tail group.

In another embodiment of the invention, including any of the foregoing embodiments, the agent comprises one or more compounds selected from the group consisting of alpha-tocopherol hydroquinone, beta-tocopherol hydroquinone, gamma-tocopherol hydroquinone, and delta-tocopherol hydroquinone. In one embodiment, the agent comprises alpha-tocopherol hydroquinone. In one embodiment, the agent comprises beta-tocopherol hydroquinone. In one embodiment, the agent comprises gamma-tocopherol hydroquinone. In one embodiment, the agent comprises delta-tocopherol hydroquinone. In some or any embodiments, the alpha, beta, gamma, and delta-tocopherol hydroquinones have the naturally-occurring stereochemistry, i.e. 3(R)-hydroxy and 7(R)-methyl and 11(R)-methyl on the tail group.

Any one or more of the compounds described herein, including all of the foregoing compounds, can be used in a composition comprising a pharmaceutically acceptable carrier, pharmaceutically acceptable excipient, or pharmaceutically acceptable vehicle. Any one or more of the compounds described herein, including all of the foregoing compounds, can be formulated into a unit dose formulation.

For all the compounds, compositions, formulations and methods described herein, any compound, composition, or formulation in the quinone form can also be used in its reduced form (hydroquinone) when desired. That is, the compounds recited herein as cyclohexadienedione compounds (oxidized quinone) form can also be used in their benzenediol (reduced hydroquinone) form as desired.

For all compounds, compositions, and formulations described herein, and all methods using a compound or composition or formulation described herein, the compounds or compositions can either comprise the listed components or steps, or can “consist essentially of” the listed components or steps, or can “consist of” the listed components or steps. That is, the transitional phrase “comprising” or “comprises” can be replaced by the transitional phrase “consisting essentially of” or “consists essentially of” Alternatively, the transitional phrase “comprising” or “comprises” can be replaced, in some or any embodiments, by the transitional phrase “consisting of” or “consists of” When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, excipients, or diluents and other such components which do not substantially affect the condition being treated.

The present invention provides, in some embodiments, methods of screening for identifying molecules capable of modulating the activity of a sulfide:quinone oxidoreductase. The sulfide:quinone oxidoreductase can be any sulfide:quinone oxidoreductase known to those of skill, including any sulfide:quinone oxidoreductase provided herein. The methods comprise the steps of contacting a sulfide:quinone oxidoreductase or a cell expressing a sulfide:quinone oxidoreductase with a test molecule and measuring at least one activity of the sulfide:quinone oxidoreductase. Molecules that modulate sulfide:quinone oxidoreductase activity are identified as molecules capable of modulating its activity. Molecules identified by the screening assays can be administered to a subject for any method of treatment or prevention described herein. Exemplary test molecules include small organic molecules (e.g., with a molecular weight less than 1 kD), that may be obtained by organic synthesis or combinatorial chemistry; nucleic acids and proteins.

### Mods for Carrying out the Invention

The present invention provides compounds and compositions for use in increasing thiosulfate, decreasing hydrogen sulfide, and/or preventing an increase in hydrogen sulfide in a subject, a tissue and/or a cell.

The abbreviations used herein have their conventional meaning within the chemical and biological arts, unless otherwise specified.

Reference to “about” a value or parameter herein includes (and describes) variations that are directed to that value or parameter per se. For example, description referring to “about X” includes description of “X”.

The terms “a” or “an,” as used in herein means one or more, unless the context clearly dictates otherwise.

By “subject,” “individual,” or “patient” is meant an individual organism, preferably a vertebrate, more preferably a mammal, most preferably a human. In some examples, the subject has a certain condition, such as but not limited to, a decreased metabolic rate. In other non-limiting examples, the subject has been exposed to H₂S. In other non-limiting examples, the subject has a tissue which includes a ratio of [H₂S]/[S₂O₃²⁻] of 0.5 or more. In other non-limiting examples, the subject has a tissue which includes a ratio of [H₂S]/[S₂O₃²⁻] of 0.6 or more. In other non-limiting examples, the subject has a tissue which includes a ratio of [H₂S]/[S₂O₃²⁻] of 0.7 or more. In other non-limiting examples, the subject is in need of treatment with a modulator of SQOR. In other non-limiting examples, the subject has been exposed to an inhibitor of SQOR. In other non-limiting examples, the subject has been exposed to an agonist of SQOR. In other non-limiting examples, the subject has been exposed to an antagonist of SQOR. In other non-limiting examples, the subject is in need of treatment with an activator of SQOR. In other non-limiting examples, the subject has a tissue which includes 20 nM or less thiosulfate.

“Tissue” indicates tissue in or from a vertebrate, more preferably a mammal, most preferably a human.

In the context of increasing a thiosulfate level, an “effective amount” of a compound is an amount of the compound sufficient to increase a thiosulfate level in a subject, a tissue, or a cell. In some embodiments, an effective amount means preventing, suppressing, eliminating, ameliorating, retarding progression of, or decreasing one or more symptoms of a disorder that is caused by a low thiosulfate level, or that may be treated by thiosulfate, in a cell, tissue, or subject that has received one or more compounds or compositions disclosed herein, compared to a cell, tissue, or subject that has not received one or more compounds or compositions disclosed herein. The effective amount may be delivered in one or more doses. The effective amount may in some embodiments be delivered systemically to the subject. In some embodiments, the effective amount may be delivered locally to a tissue of interest, or to an isolated tissue.

In the context of decreasing hydrogen sulfide or preventing an increase in hydrogen sulfide, an “effective amount” of a compound is an amount of the compound sufficient to lower a H₂S level, or prevent an increase in a H₂S level in a subject, tissue, or cell. The effective amount may be delivered in one or more doses. The effective amount may in some embodiments be delivered systemically to the individual. In some embodiments, the effective amount may be delivered locally to a tissue of interest, or to an isolated tissue. The H₂S level(s) may be measured in the matrix of interest (e.g. cells, plasma, whole blood, or tissues (e.g. tissues of disease pathology)) by derivatization of H₂S as the bis-bromobimane adduct of H₂S. See e.g. Shen, X., et al., Measurement of plasma hydrogen sulfide in vivo and in vitro, Free Radical Biology and Medicine 50(2011), pp 1021-1031. In some embodiments, an “effective amount” is sufficient to prevent, reduce, eliminate, retard progression of, or reduce the severity of a disorder or one or more symptoms of a disorder related to an increased H₂S level, or one or more symptoms related to an increased H₂S level.

In the context of methods for inducing a temporarily decreased metabolic state in an individual, tissue, or cell followed by reversing the temporarily decreased metabolic state, an “effective amount” is sufficient to at least partially reverse one or more symptoms of the decreased metabolic state in the individual or tissue. The effective amount may be delivered in one or more doses. The effective amount may in some embodiments be delivered systemically to the individual. In some embodiments, the effective amount may be delivered locally to a tissue of interest, or to an isolated tissue.

A “therapeutically effective amount” of a compound is an amount of the compound, which, when administered to a subject, is sufficient to reduce or eliminate either a disorder or one or more symptoms of a disorder, or to retard the progression of a disorder or of one or more symptoms of a disorder, or to reduce the severity of a disorder or of one or more symptoms of a disorder, or to suppress the clinical manifestation of a disorder, or to suppress the manifestation of adverse symptoms of a disorder. A therapeutically effective amount can be given in one or more administrations.

A “prophylactically effective amount” of a compound is an amount of the compound, which, when administered to a subject prior to onset of the disorder, is sufficient to suppress the deleterious effects of the disorder, or the clinical manifestation of the disorder, or to suppress the manifestation of adverse symptoms of the disorder. A prophylactically effective amount can be given in one or more administrations.

An “agent” is a compound suitable for increasing thiosulfate, decreasing hydrogen sulfide, and/or preventing an increase in hydrogen sulfide in a subject, tissue, or cell. In some or any embodiments, the agent is one or more compounds or compositions of Formula A, Formula I, Formula I-Unsat, Formula I-Sat, Formula II, Formula II-Unsat, Formula II-Sat, Formula III, Formula III-Unsat, Formula III-Sat, Formula IV, Formula IV-Unsat, Formula IV-Unsat-R, Formula IV-Unsat-S, Formula IV-Sat, Formula IV-Sat-R, Formula IV-Sat-S, Formula V, Formula V-Unsat, Formula V-Sat, Formula VI, Formula VI-Unsat, Formula VI-Unsat-R, Formula VI-Unsat-S, Formula VI-Sat, Formula VI-Sat-R, and Formula VI-Sat-S, or a hydroquinone of any of those formulas, and tocotrienol quinones, tocotrienol hydroquinones, tocopherol quinones, and tocopherol hydroquinones disclosed herein, or a stereoisomer, mixtures of stereoisomers, prodrug, salt, phosphate substituted form, crystalline form, non-crystalline form, isotopologue, deuterated form, hydrate, or solvate thereof

As used herein, the term “sulfide:quinone oxidoreductase” “SQOR” or “SQR” refers to a mitochondrial enzyme capable of catalyzing a 2-electron transfer from H₂S to a quinone, generally consuming SO₃²⁻ and producing a hydroquinone and S₂O₃²⁻ (in some embodiments catalyzing a 2-electron transfer from H₂S to a quinone, generally consuming H₂SO₃ and producing a hydroquinone and H₂S₂O₃). Sulfide:quinone oxidoreductase enzymes belong to the enzyme commission (EC) number 1.8.5.4. In certain embodiments, the term refers to the enzyme with an FAD cofactor. In some examples, provided herein are useful sulfide:quinone oxidoreductases which include wild-type sulfide: quinone oxidoreductase and variant sulfide:quinone oxidoreductases having up to ten amino acid substitutions or up to ten conservative amino acid substitutions.

The embodiments described herein, unless otherwise indicated, employ standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5″ edition, Garland Science, New York, N.Y., 2008; Voet, D. et al. “Fundamentals of Biochemistry: Life at the Molecular Level,” 3rd edition, John Wiley & Sons, Hoboken, N.J., 2008; Sambrook, J. et al., “Molecular Cloning: A Laboratory Manual,” 3rd edition, Cold Spring Harbor Laboratory Press, 2001; Ausubel, F. et al., “Current Protocols in Molecular Biology,” John Wiley & Sons, New York, 1987 and periodic updates; Freshney, RI., “Culture of Animal Cells: A Manual of Basic Technique,” 4th edition, John Wiley & Sons, Somerset, N.J., 2000; and the series “Methods in Enzymology,” Academic Press, San Diego, Calif.

### Compounds for Use in the Invention

The compounds disclosed below of Formula A, Formula I, Formula I-Unsat, Formula I-Sat, Formula II, Formula II-Unsat, Formula II-Sat, Formula III, Formula III-Unsat, Formula III-Sat, Formula IV, Formula IV-Unsat, Formula IV-Unsat-R, Formula IV-Unsat-S, Formula IV-Sat, Formula IV-Sat-R, Formula IV-Sat-S, Formula V, Formula V-Unsat, Formula V-Sat, Formula VI, Formula VI-Unsat, Formula VI-Unsat-R, Formula VI-Unsat-S, Formula VI-Sat, Formula VI-Sat-R, and Formula VI-Sat-S, or a hydroquinone of any of those formulas, and the various tocotrienol quinones, tocotrienol hydroquinones, tocopherol quinones, and tocopherol hydroquinones described below, can be used as agents as described herein.

Compounds for use in the invention include one or more compounds of Formula A:

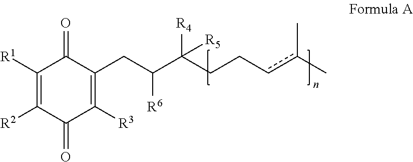

wherein:
each bond indicated with a dashed line, independently of the other bonds indicated with a dashed line, can be a single bond or a double bond;
n is 1, 2, or 3;
R¹, R², and R³ are independently selected from H, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, n is 1.
In some embodiments, n is 2. In some embodiments, n is 3.

Compounds for use in the invention include one or more compounds of Formula I:

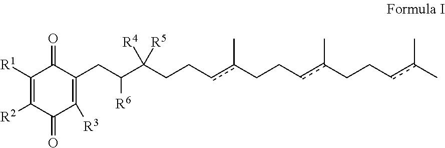

wherein:
each bond indicated with a dashed line, independently of the other bonds indicated with a dashed line, can be a single bond or a double bond;
R¹, R², and R³ are independently selected from H, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula I-Unsat:

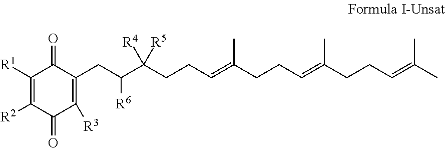

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula I-Sat:

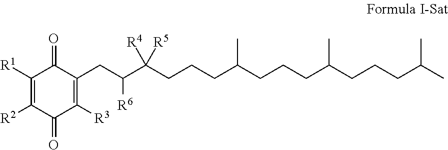

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof

Compounds for use in the invention also include one or more compounds of Formula II:

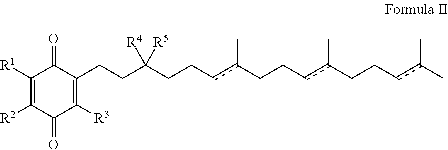

where the bonds and substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof

Compounds for use in the invention also include one or more compounds of Formula II-Unsat:

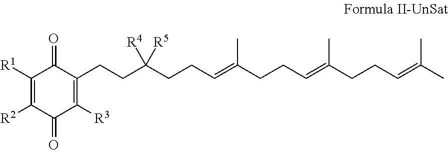

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula II-Sat:

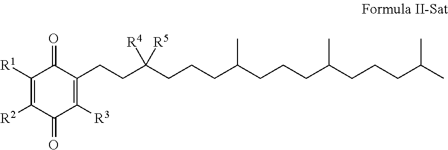

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula III:

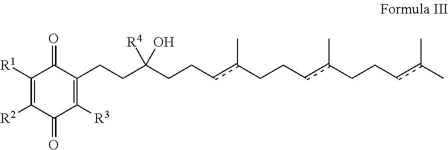

where the bonds and substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula III-Unsat:

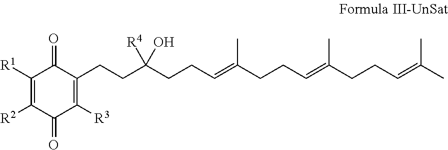

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula III-Sat:

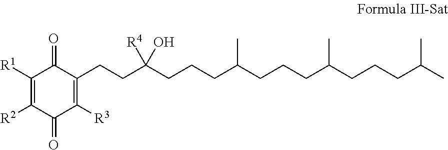

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof.

Compounds for use in the invention also include one or more compounds of Formula IV:

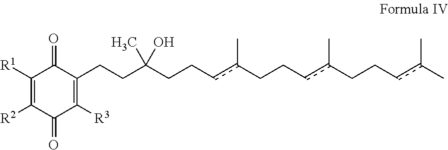

where the bonds and substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, the compounds of Formula IV are compounds of Formula IV-R:

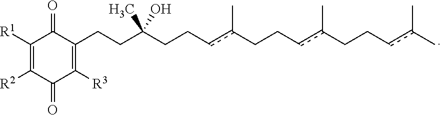

In some embodiments, the compounds of Formula IV are compounds of Formula IV-S:

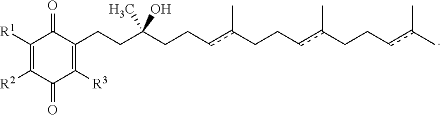

Compounds for use in the invention also include one or more compounds of Formula IV-Unsat:

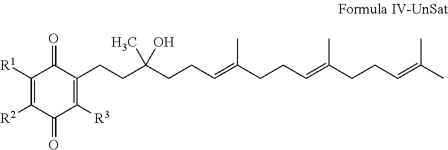

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, the compounds of Formula IV-Unsat are compounds of Formula IV-Unsat-R:

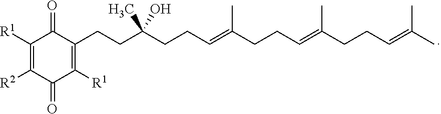

In some embodiments, the compounds of Formula IV-Unsat are compounds of Formula IV-Unsat-S:

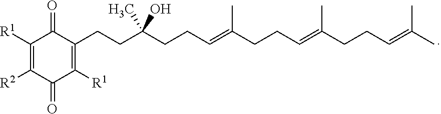

Compounds for use in the invention also include one or more compounds of Formula IV-Sat:

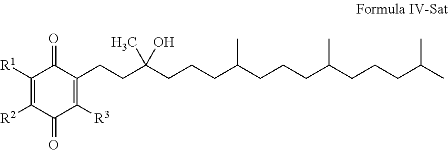

where the substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, the compounds of Formula IV-Sat are compounds of Formula IV-Sat-R:

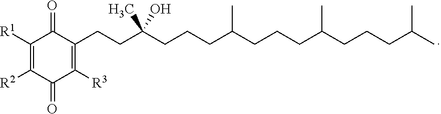

In some embodiments, the compounds of Formula IV-Sat are compounds of Formula IV-Sat-S:

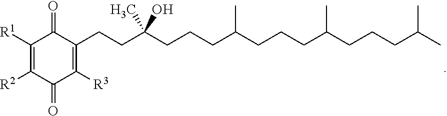

Compounds for use in the invention also include one or more compounds of Formula V:

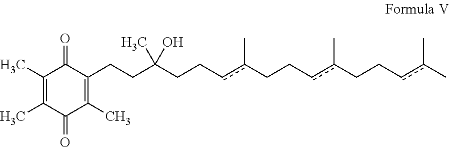

where the bonds and substituents are as indicated for Formula I; or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, the compounds of Formula V are compounds of Formula V-R:

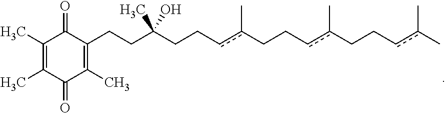

In some embodiments, the compounds of Formula V are compounds of Formula V-S:

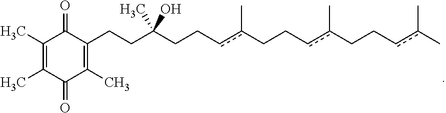

Compounds for use in the invention also include one or more compounds of Formula V-Unsat:

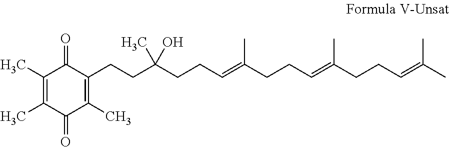 [SMILES: CC(C)=CCC/C(C)=C/CC/C(C)=C/CCC(C)(O)CCC1=C(C)C(=O)C(C)=C(C)C1=O]

which is alpha-tocotrienol quinone; or a stereoisomer, mixtures of stereoisomers, salt, phosphate substituted form, crystalline form, non-crystalline form, isotopologue, deuterated form, hydrate, or solvate thereof; or the hydroquinone form thereof. In some embodiments, the compound of Formula V-Unsat is 3R-alpha-tocotrienol quinone. In some embodiments, the compound of Formula V-Unsat is 3S-alpha-tocotrienol quinone.

Compounds for use in the invention also include one or more compounds of Formula V-Sat:

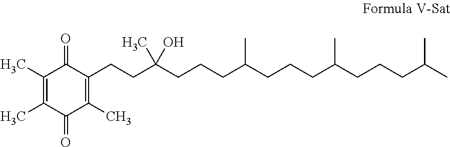 [SMILES: CC1=C(C)C(=O)C(CCC(C)(O)CCCC(C)CCCC(C)CCCC(C)C)=C(C)C1=O]

which is alpha-tocopherol quinone; or a stereoisomer, mixtures of stereoisomers, salt, phosphate substituted form, crystalline form, non-crystalline form, isotopologue, deuterated form, hydrate, or solvate thereof; or the hydroquinone form thereof. In some embodiments, the compound of Formula V-Sat is (3R,7R,11R)-alpha-tocopherol quinone. In some embodiments, the compound of Formula V-Sat is (3S,7R,11R)-alpha-tocopherol quinone. In some embodiments, the compound of Formula V-Sat is (3R,7S,11R)-alpha-tocopherol quinone. In some embodiments, the compound of Formula V-Sat is (3S,7S,11R)-alpha-tocopherol quinone. In some embodiments, the compound of Formula V-Sat is (3R,7R,11S)-alpha-tocopherol quinone. In some embodiments, the compound of Formula V-Sat is (3S,7R,11S)-alpha-tocopherol quinone. In some embodiments, the compound of Formula V-Sat is (3R,7S,11S)-alpha-tocopherol quinone. In some embodiments, the compound of Formula V-Sat is (3S,7S,11S)-alpha-tocopherol quinone.

Compounds for use in the invention also include one or more compounds of Formula VI:

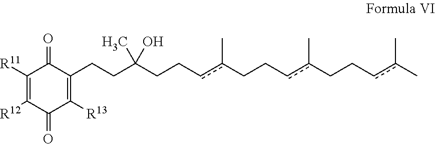

where R¹¹, R^{12,} and R¹³ are independently selected from the group consisting of —CH₃ and —H;
or a stereoisomer, mixtures of stereoisomers, salt, phosphate substituted form, crystalline form, non-crystalline form, isotopologue, deuterated form, hydrate, or solvate thereof; or the hydroquinone form thereof. In some embodiments, the compounds of Formula VI are compounds of Formula VI-R:

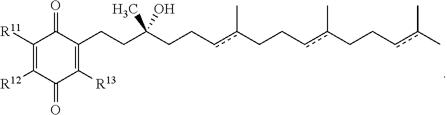

In some embodiments, the compounds of Formula VI are compounds of Formula VI-S:

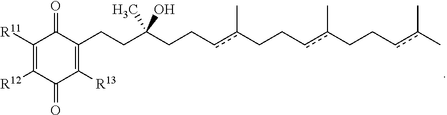

Compounds for use in the invention also include one or more compounds of Formula VI-Unsat:

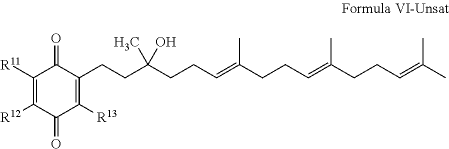

where the substituents are as indicated for formula VI; or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, the compounds of Formula VI-Unsat are compounds of Formula VI-Unsat-R:

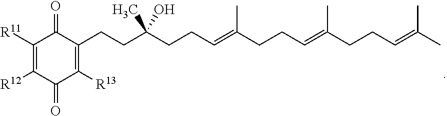

In some embodiments, the compounds of Formula VI-Unsat are compounds of Formula VI-Unsat-S:

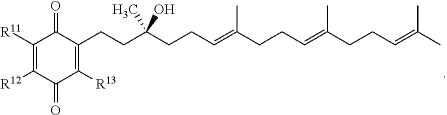

Compounds for use in the invention also include one or more compounds of Formula VI-Sat:

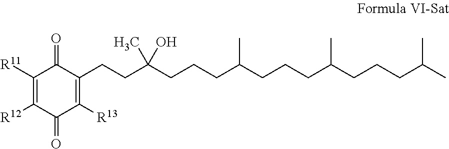

where the substituents are as indicated for formula VI; or a stereoisomer, mixtures of stereoisomers, salt, phosphate substituted form, crystalline form, non-crystalline form, isotopologue, deuterated form, hydrate, or solvate thereof;
or the hydroquinone form thereof. In some embodiments, the compounds of Formula VI-Sat are compounds of Formula VI-Sat-R:

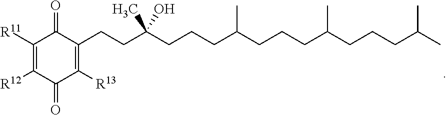

In some embodiments, the compounds of Formula VI-Sat are compounds of Formula VI-Sat-S:

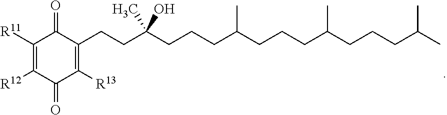

In other embodiments of the invention, the agent comprises one or more compounds selected from the group consisting of alpha-tocotrienol quinone, beta-tocotrienol quinone, gamma-tocotrienol quinone, and delta-tocotrienol quinone. In one embodiment, the agent comprises alpha-tocotrienol quinone. In one embodiment, the agent comprises beta-tocotrienol quinone. In one embodiment, the agent comprises gamma-tocotrienol quinone. In one embodiment, the agent comprises delta-tocotrienol quinone.

In another embodiment of the invention, the agent comprises one or more compounds selected from the group consisting of alpha-tocotrienol hydroquinone, beta-tocotrienol hydroquinone, gamma-tocotrienol hydroquinone, and delta-tocotrienol hydroquinone. In one embodiment, the agent comprises alpha-tocotrienol hydroquinone. In one embodiment, the agent comprises beta-tocotrienol hydroquinone. In one embodiment, the agent comprises gamma-tocotrienol hydroquinone. In one embodiment, the agent comprises delta-tocotrienol hydroquinone.

In another embodiment of the invention, the agent comprises one or more compounds selected from the group consisting of alpha-tocopherol quinone, beta-tocopherol quinone, gamma-tocopherol quinone, and delta-tocopherol quinone. In one embodiment, the agent comprises alpha-tocopherol quinone. In one embodiment, the agent comprises beta-tocopherol quinone. In one embodiment, the agent comprises gamma-tocopherol quinone. In one embodiment, the agent comprises delta-tocopherol quinone.

In another embodiment of the invention, the agent comprises one or more compounds selected from the group consisting of alpha-tocopherol hydroquinone, beta-tocopherol hydroquinone, gamma-tocopherol hydroquinone, and delta-tocopherol hydroquinone. In one embodiment, the agent comprises alpha-tocopherol hydroquinone. In one embodiment, the agent comprises beta-tocopherol hydroquinone. In one embodiment, the agent comprises gamma-tocopherol hydroquinone. In one embodiment, the agent comprises delta-tocopherol hydroquinone.

For all the formulations and methods described herein, any composition in the quinone form can also be used in its reduced form (hydroquinone) when desired. That is, the compounds recited herein as cyclohexadienedione compounds (oxidized quinone) form can also be used in their benzenediol (reduced hydroquinone) form as desired.

While the compounds described herein can occur and can be used as the neutral (non-salt) compound, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. In some embodiments, inorganic acids include, but are not limited to, hydrochloric acid, hydrobromic acid, sulfuric acid, nitric acid, and phosphoric acid. In some embodiments, organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. In some embodiments, inorganic salts of acid compounds include, but are not limited to, alkali metal and alkaline earth salts, such as sodium salts, potassium salts, magnesium salts, and calcium salts; ammonium salts; and aluminum salts. In some embodiments, organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared.

The invention also includes all stereoisomers of the compounds, including diastereomers and enantiomers. The invention also includes mixtures of stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The compounds can be administered in prodrug form. Prodrugs are derivatives of the compounds, which are themselves relatively inactive but which convert into the active compound when introduced into the subject in which they are used by a chemical or biological process in vivo, such as an enzymatic conversion. Suitable prodrug formulations include, but are not limited to, peptide conjugates of the compounds of the invention and esters of compounds of the inventions. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.), Washington : The Academy, 1977.

“C₁-C₄ alkyl” is intended to embrace a saturated linear, branched, cyclic, or a combination thereof, hydrocarbon of 1 to 4 carbon atoms. In some embodiments of “C₁-C₄ alkyl” are methyl, ethyl, n-propyl, isopropyl, cyclopropyl, n-butyl, isobutyl, sec-butyl, t-butyl, cyclobutyl, cyclopropyl-methyl, and methyl-cyclopropyl.

“Halogen” or “halo” designates fluoro, chloro, bromo, and iodo.

“C₁-C₄ haloalkyl” is intended to embrace any C₁-C₄ alkyl substituent having at least one halogen substituent, in some embodiments 1 to 6 halogens, in some embodiments, 1 to 3 halogens; the halogen can be attached via any valence on the C₁-C₄ alkyl group. In some embodiments of C₁-C₄ haloalkyl is —CF₃, —CCl₃, —CHF₂, —CHCl₂, —CHBr₂, —CH₂F, —CH₂Cl.

“Deuterated form” means the compound is isotopically enriched for deuterium in at least one atom.

“Isotopologue” means a compound which differs, i.e. in the number of neutrons, in its isotopic composition of at least one atom from the parent molecule having a natural isotopic composition. In some or any embodiments, the compound is isotopically enriched.

The term “isotopic composition,” as used herein, and unless otherwise specified, refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopic enriched,” as used herein, and unless otherwise specified, refers to the percentage of incorporation of an amount of a specific isotope at a given atom in a molecule in the place of that atom's natural isotopic abundance. In certain embodiments, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

The term “isotopically enriched,” as used herein, and unless otherwise specified, refers to an atom having an isotopic composition other than the natural isotopic composition of that atom. “Isotopically enriched” may also refer to a compound containing at least one atom having an isotopic composition other than the natural isotopic composition of that atom.

“Phosphate substituted form” means that any unsubstituted hydroxy group of the compound is substituted with a phosphate group, —P(O)(OH)₂ or —PO₃²⁻,

### Increasing Thiosulfate Levels

Diseases or disorders which may be treated by thiosulfate administration or which are caused by low thiosulfate may also be treated by the methods of the invention. Non-limiting examples include disorders relating to or caused by or exacerbated by oxidative stress, antidoting cyanide poisoning, preserving renal function, treatment of acute lung injury, and treatment or prevention of calciphylaxis in blood vessels, kidney toxicity in cancer therapy, antibacterial infection, anti- fungal infection, ulcerative colitis, hypertension, and proteinuria.

One skilled in the art would be able to determine the appropriate amount or dosage of the compound required to achieve the intended effect.

### Modulation of Hydrogen Sulfide Levels

The inventors have surprisingly discovered that certain redox-active compounds are useful in oxidizing hydrogen sulfide (H₂S), converting H₂S into, for example, HSSH, thiosulfate, or other species. These compounds may be used in various applications wherein reducing or preventing an increased H₂S level is desired.

For example, exposure to increased H₂S in mammals, whether from exogeneous sources or from internal normal or abnormal biological processes, results in symptoms ranging from lethargy, decreased heart, respiration rate and/or body temperature, hibernation, neurological symptoms mimicking those found in Leigh's Syndrome, and death. The compounds of the invention may be useful in treating or preventing disorders, or treating or preventing one or more symptoms of a disorder associated with an increased H₂S level, such as stroke, or disorders involving abnormal ETHE1 (persulfide dioxygenase) or sulfide oxidase activity. In addition, the compounds of the invention may be useful in treating a patient exposed to exogeneous H₂S or preventing illness in an individual who may be exposed to exogeneous H₂S.

In other embodiments, an individual, or a portion of an individual (e.g. a tissue or organ), may have been deliberately exposed to H₂S. For example, H₂S has been shown in mice to reduce heart rate, respiration, and body temperature. H₂S may be useful in inducing hibernation and/or a greatly decreased metabolic state in an animal and/or portion of an animal. In non-limiting examples, H₂S may be used in trauma care (e.g. to slow metabolism during transport, evaluation, and treatment of a traumatic injury or organ transplantation); in mine disasters, fires or other situations in which lowering the requirement for O₂ by slowing respiration rate may be useful; suspended animation; or any other situation in which reducing metabolism/respiration would be advantageous. The compounds of the invention may be useful in counteracting this state, for example in a trauma case, bringing the patient back to normal metabolic rate once the traumatic injury has been addressed, or bringing an animal out of hibernation, etc.

The compounds may be administered as appropriate for the intended method. For example, in methods of reducing H₂S levels in a patient, the compound may be administered systemically to the patient, or locally to one or more of the affected tissues.

One skilled in the art would be able to determine the appropriate amount or dosage of the compound required to achieve the intended effect.

### Hydrogen Sulfide Measurement and Plasma Levels of Hydrogen Sulfide

Levels of hydrogen sulfide can be measured in human plasma by methods disclosed in International Patent Appl. No. WO 2013/14826; in Shen X. et al., “Hydrogen sulfide measurement using sulfide dibimane: critical evaluation with electrospray ion trap mass spectrometry,” Nitric Oxide 41:97-104 (2014); and Revsbech, I. et al., “Hydrogen sulfide and nitric oxide metabolites in the blood of free-ranging brown bears and their potential roles in hibernation,” Free Radical Biology and Medicine 73:349-357 (2014). Quantitative mass spectroscopy using bromobimane reagent and internal standards allows accurate determination of hydrogen sulfide levels in human plasma. Similar methods may be used for measuring thiosulfate levels.

Typical levels of hydrogen sulfide in human plasma range between 1 nanomolar to 20 nanomolar. Levels of hydrogen sulfide above 20 nM are deleterious, and indicate that the individual is a candidate for reduction of hydrogen sulfide levels using the compounds and methods disclosed in the present invention.

### Pharmaceutical Formulations

The compounds described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, in some embodiments, calcium phosphate, magnesium stearate, talc, monosaccharides, disaccharides, starch, gelatin, cellulose, methyl cellulose, sodium carboxy methyl cellulose, dextrose, hydroxypropyl-β-cyclodextrin, polyvinylpyrrolidone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic or prophylactic effect.

Pharmaceutical compositions containing the compounds of the invention may be in any form suitable for the intended method of administration, including, in some embodiments, a solution, a suspension, or an emulsion. Liquid carriers are typically used in preparing solutions, suspensions, and emulsions. Liquid carriers contemplated for use in the practice of the present invention include, in some embodiments, water, saline, pharmaceutically acceptable organic solvent(s), pharmaceutically acceptable oils or fats, and the like, as well as mixtures of two or more thereof The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, in some embodiments, monohydric alcohols, such as ethanol, and polyhydric alcohols, such as glycols. Suitable oils include, in some embodiments, sesame oil, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, in some embodiments, a biodegradable material that can degrade spontaneously in situ and in vivo, in some embodiments, by hydrolysis or enzymatic cleavage, e.g., by proteases. The delivery system may be, in some embodiments, a naturally occurring or synthetic polymer or copolymer, in some embodiments, in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. In some embodiments, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous, intravenous, intramuscular, and intrasternal injection or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, in some embodiments, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, in some embodiments, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

The formulations of the present invention may comprise two or more compounds or compositions as described herein.

The invention also provides articles of manufacture and kits containing materials useful for the methods described herein.

The amount of active ingredient that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, and diet of the patient; time of administration, route of administration, rate of excretion, or drug combination; and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount or prophylactically effective amount for a given situation can be readily determined by routine experimentation and is within the skill and judgment of the ordinary clinician.

The single or multiple dosages which can be used include an amount independently selected from about 0.1 mg/kg to about 600 mg/kg body weight, or about 1.0 mg/kg to about 500 mg/kg body weight, or about 1.0 mg/kg to about 400 mg/kg body weight, or about 1.0 mg/kg to about 300 mg/kg body weight, or about 1.0 mg/kg to about 200 mg/kg body weight, or about 1.0 mg/kg to about 100 mg/kg body weight, or about 1.0 mg/kg to about 50 mg/kg body weight, or about 1.0 mg/kg to about 30 mg/kg body weight, or about 1.0 mg/kg to about 10 mg/kg body weight, or about 10 mg/kg to about 600 mg/kg body weight, or about 10 mg/kg to about 500 mg/kg body weight, or about 10 mg/kg to about 400 mg/kg body weight, or about 10 mg/kg to about 300 mg/kg body weight, or about 10 mg/kg to about 200 mg/kg body weight, or about 10 mg/kg to about 100 mg/kg body weight, or about 50 mg/kg to about 150 mg/kg body weight, or about 100 mg/kg to about 200 mg/kg body weight, or about 150 mg/kg to about 250 mg/kg body weight, or about 200 mg/kg to about 300 mg/kg body weight, or about 250 mg/kg to about 350 mg/kg body weight, or about 200 mg/kg to about 400 mg/kg body weight, or about 300 mg/kg to about 400 mg/kg body weight, or about 250 mg/kg to about 300 mg/kg body weight, or about 300 mg/kg body weight. Compounds of the present invention may be administered in a single daily dose, or the total daily dosage may be administered in divided dosage of two, three or four times daily.

Single or multiple doses can be administered. In some embodiments, the dose is administered once, twice, three times, four times, five times, or six times. In some embodiments, the dose is administered once per day, twice per day, three times per day, or four times per day. In some embodiments, the dose is administered every hour, every two hours, every three hours, every four hours, every 6 hours, every 12 hours, or every 24 hours.

In some embodiments, the amount of compound administered to the patient is sufficient to result in a plasma concentration of about 10 nM to about 10 micromolar.

While the compounds of the invention can be administered as the sole active pharmaceutical agent, they can also be used in combination with one or more other agents. In some embodiments, the compound(s) of the invention are administered as the sole active pharmaceutical agent that is present in a therapeutically effective amount.

When additional active agents are used in combination with the compounds of the present invention, the additional active agents may generally be employed in therapeutic amounts as indicated in the Physicians' Desk Reference (PDR) 53rd Edition (1999), or such therapeutically useful amounts as would be known to one of ordinary skill in the art.

The compounds of the invention and the other therapeutically active agents or prophylactically effective agents can be administered at the recommended maximum clinical dosage or at lower doses. Dosage levels of the active compounds in the compositions of the invention may be varied so as to obtain a desired response depending on the route of administration, severity of the disease and the response of the patient. When administered in combination with other therapeutic or prophylactic agents, the therapeutic agents or prophylactic agents can be formulated as separate compositions that are given at the same time or different times, or the therapeutic agents or prophylactic agents can be given as a single composition.

### Preparation of Compounds of the Invention

In general, the nomenclature used in this Application was generated with the help of naming package within the ChemOffice®. version 11.0 suite of programs by CambridgeSoft Corp (Cambridge, Mass.).

The compounds of this invention can be prepared from readily available starting materials using general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Preparation of the compounds disclosed herein is described in co-assigned US Patent Application Publications No. 2006/0281809 and 2010/0105930.

Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, in some embodiments, optically active starting materials or stereoselective reagents well-known in the art. Alternatively, racemic mixtures of such compounds ca be separated using, in some embodiments, chiral column chromatography, chiral resolving agents and the like.

### Modulators of Sulfide:Quinone Oxidoreductase

In some examples, provided herein are modulators of sulfide:quinone oxidoreductase which include both activators (agonists) and inhibitors (antagonists), and can be selected by using a variety of screening assays. In one embodiment, modulators can be identified by determining if a test compound binds to a sulfide:quinone oxidoreductase; wherein, if binding has occurred, the compound is a candidate modulator. The present invention provides some of these modulators. Additional tests can be carried out on such a candidate modulator. Alternatively, a candidate compound can be contacted with a sulfide:quinone oxidoreductase, and a biological activity of the enzyme can be assayed; a compound that alters the biological activity of the sulfide:quinone oxidoreductase is a modulator of a sulfide:quinone oxidoreductase. Generally, a compound that reduces a biological activity of a sulfide:quinone oxidoreductase is an inhibitor of the enzyme.

In some examples, provided herein are methods of identifying modulators of sulfide:quinone oxidoreductases include incubating a candidate compound in a cell culture containing one or more sulfide:quinone oxidoreductases and assaying one or more biological activities or characteristics of the cells. In some examples, provided herein are compounds that alter the biological activity or characteristic of the cells in the culture are potential modulators of sulfide:quinone oxidoreductases. Biological activities that can be assayed include, for example, sulfide:quinone oxidoreductase enzymatic activity (e.g., H₂S oxidation), levels of sulfide:quinone oxidoreductase, levels of mRNA encoding a sulfide:quinone oxidoreductase, and/or one or more functions specific to a sulfide: quinone oxidoreductase. In some embodiments, provided herein are separate cultures containing different levels of a sulfide:quinone oxidoreductase are contacted with a candidate compound. If a change in biological activity is observed, and if the change is greater in the culture having higher levels of sulfide:quinone oxidoreductase, the compound is identified as a modulator of a sulfide:quinone oxidoreductase. Determination of whether the compound is an activator or an inhibitor of a sulfide:quinone oxidoreductase may be apparent from the phenotype induced by the compound, or may require further assay, such as a test of the effect of the compound on sulfide:quinone oxidoreductase enzymatic activity.

In some examples, provided herein is a sulfide:quinone oxidoreductase which can be prepared or obtained , either biochemically or recombinantly, by any technique apparent to those of skill in the art. Cells expressing sulfide:quinone oxidoreductase as well cell cultures can be prepared according to techniques apparent to those of skill in the art. Useful enzymatic assays include those described herein an those known to those of skill. Exemplary preparations and assays are described, for example, in Jackson et al., 2012, Biochemistry 51:6804-6815.

In some examples, provided herein are test compounds which include, but are not limited to, small organic compounds (e.g., organic molecules having a molecular weight between about 50 and about 2,500 Da), nucleic acids, and proteins. In some examples, provided herein a compound or plurality of compounds which may be chemically synthesized or microbiologically produced and/or comprised in, for example, samples, e.g., cell extracts from, e.g., plants, animals or microorganisms. Furthermore, the compound(s) may be known in the art but hitherto not known to be capable of modulating a sulfide:quinone oxidoreductase. The reaction mixture for assaying for a modulator of a sulfide:quinone oxidoreductase can be a cell-free extract or can comprise a cell culture or tissue culture. A plurality of compounds can be, e.g., added to a reaction mixture, added to a culture medium, introduced into a cell or administered to a transgenic animal. The cell or tissue employed in the assay can be, for example, a bacterial cell, a fungal cell, an insect cell, a vertebrate cell, a mammalian cell, a primate cell, a human cell or can comprise or be obtained from a non-human transgenic animal.

In some examples, provided herein is an inhibitor of a sulfide:quinone oxidoreductase which can be a competitive inhibitor, an uncompetitive inhibitor, a mixed inhibitor or a non-competitive inhibitor. In some examples, provided herein are competitive inhibitors which often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vmax are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site.

Kinetic Parameters

Methods for determining enzymatic kinetic parameters are known to those skilled in the art. Exemplary methods are shown in the Examples below. In general, since these redox active compounds may interconvert between the reduced (e.g. hydroquinone) and oxidized (e.g. quinone) forms, in particular the possible oxidation of hydroquinone/hydroquinoid to quinone/quinoid by oxygen in solution, to limit non-enzymatic conversion of hydroquinone/hydroquinoid to quinone/quinoid and avoid subsequent complications for the enzymatic activity analysis, the assays may be performed under reduced oxygen atmosphere (e.g. in a controlled-atmosphere glovebox or screwcap cuvettes purged with nitrogen). In some embodiments, O₂ concentrations of less than 1000 ppm are used for these assays. In some embodiments, single-digit ppm O₂ concentrations are used for these assays.

Briefly, for kinetic parameter assays, SQOR enzyme, compound, inorganic sulfide (e.g. H₂S, HS⁻, and S²⁻) and a sulfur acceptor (including, but are not limited to: sulfite ion (SO₃²⁻), cyanide ion (CN⁻), and alkyl thiolates (e.g. glutathione)) are brought together. The reduction of the quinone/quinoid substrate (compound) to hydroquinone/hydroquinoid product in the presence of sulfide ion, sulfur acceptor and SQOR enzyme may be monitored by the decrease in quinone/quinoid concentration over time, e.g. by UV, fluorescence, mass spectrometry, (U)HPLC with UV-visible or electrochemical detection, enzyme coupled assays with UV-visible or fluorescence readout and detection of quinone derivatives by any of the above or other methods after reaction with derivatizing agents including phenylhydrazones. Assays can be performed in e.g. screwcap cuvettes purged with nitrogen or in plate format in a controlled atmosphere chamber in volumes from approximately 50-1000 μL, provided the pathlengths are adjusted accordingly.

The kinetic parameters of compounds with SQOR may be determined by varying the concentration of the compound over a suitable range and interrogating the impact of that variation on the enzymatic rate. The parameters k_{cat} and Kₘ, for example, can be determined by nonlinear fitting of the rate data to the Michaelis-Menten equation:

Rate=k_{cat}*[S]/(Kₘ+[S])

or a derivative, where [S] is the concentration of the substrate (here, the redox-active quinone, quinoid, hydroquinone, or hydroquinoid). These kinetic parameters individually and combined into the “specificity constant” (a second-order rate constant) k_{cat}/Kₘ provide information regarding the interactions between the substrate and enzyme. In particular, relatively large values of k_{cat}/Kₘ (exceeding ˜10⁴ M⁻¹s⁻¹), suggest compounds with potential to significantly influence SQOR activity.

Further examples of kinetic parameter determination for SQOR may be found in Jackson, M. R., et al. “Human Sulfide:Quinone Oxidoreductase Catalyzes the First Step in Hydrogen Sulfide Metabolism and Produces a Sulfane Sulfur Metabolite”, Biochemistry, Publication Date (Web): 1 Aug 2012.

The invention is further described by the following non-limiting examples and embodiments.

### EXAMPLE 1

### Quantitation of Thiosulfate in Plasma

EPI-743 has the following structure as noted in Enns, G. M., et al., “Initial Experience in the Treatment of Inherited Mitochondrial Disease with EPI-743,” Molecular Genetics and Metabolism (Impact Factor: 2.63). 10/2011; 105(1):91-102. DOI: 10.1016/j.ymgme.2011.10.009,

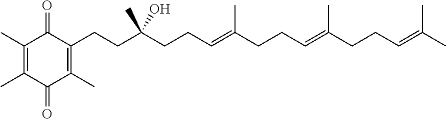 [SMILES: CC(C)=CCC/C(C)=C/CC/C(C)=C/CC[C@@](C)(O)CCC1=C(C)C(=O)C(C)=C(C)C1=O]

the entire contents of which are herein incorporated by reference in its entirety for all purposes.

As part of the Edison EPI743-12-002 Leigh Trial, 17 Leigh Syndrome patients were treated with EPI-743 (15 mg/kg up to 200 mg three times a day) for 12 months. An additional 14 Leigh Syndrome patients received placebo for the first six months, and were randomized to either 5 or 15 mg/kg up to 200 mg three times a day EPI-743 for the subsequent six months.

Plasma thiosulfate concentrations were measured at various time points, over a 12 month period, for patients on drug and placebo. Plasma samples were collected from Leigh Syndrome patients, and the samples processed according to the procedure described in Example 2.

Results

Thiosulfate levels were measured according to the procedure described in Example 2. As measured across all time points, a greater than 100% increase in mean plasma thiosulfate was observed in subjects treated with EPI-743 (from 0.21 μM (baseline) to 0.54 μM), and virtually no change in levels in placebo subjects (0.41 μM (baseline) to 0.40 μM).

### EXAMPLE 2

### Quantitation of H₂S, Thiosulfate, and Glutathione Persulfide in Plasma Samples

A quantitative method for the analysis of H₂S, thiosulfate, and glutathione persulfide in biological matrices via liquid chromatography coupled to atmospheric pressure ionization (API) tandem mass spectrometry (LC-MS/MS) is provided.

### Sample Collections and Processing Method

Patient plasma samples were collected with informed consent from the patients described in Example 1. The sulfur containing compounds were derivatized with bromo bimane to stabilize the samples for measurement. Samples were stored at −80° C. and shipped for analysis to Edison under dry ice.

The samples were analyzed by measuring the absolute concentration of H₂S, thiosulfate, and glutathione persulfide using 6-point calibration curves. The biomarker assay was run using guidelines from the FDA Bioanalytical Method Validation Guidance for Industry (2001) and the EMA Guideline on Bioanalytical Method Validation (2012).

### LC-MS/MS Parameters.

An API mass spectrometer (AB6500 Qtrap) coupled to a high performance liquid chromatograph (Eksigent MicroLC 200 Plus) was used to quantify H₂S, thiosulfate, and glutathione persulfide and their internal standards from all biological fluids and calibrants. LC separations were achieved with reversed phase chromatography using a Thermo Hypersil Gold C18 analytical column with gradient elution.

Processed biological samples were removed from −80° C. storage and thawed at 4° C. Once thawed, samples were vortexed vigorously for 1 minute. Processed biological samples were centrifuged at 4° C. with 3220 RCF for 10 minutes. Aliquots (5-50 μL) of samples were transferred to 96 well plates. Once added, the solution was then pipette mixed.

The LC system was coupled to the mass spectrometer via a Turbo-Ion Spray inlet. Source settings were optimized for analyte sensitivity at the working flow rate to ensure efficient transmission of parent ions into the mass spectrometer. The 96 well plate was then place in the HPLC autosampler and run on the Mass Spectrometer. Detection of analytes was achieved using MRM settings in positive ionization mode.

LC Settings*

| Mobile Phase A | H₂O with 0.1% formic acid | |
|---|---|---|
| Mobile Phase B | ACN with 0.1% formic acid | |
| Flow Rate | 0.4 | mL/min |
| Column Temperature | 45° | C. |
| Autosampler Temperature | 5° | C. |
| Injection Volume | 1-5 | μL |

| | |
|---|---|
| Elution Profile | Gradient: 1 min at 1% B, 3 mins |
| | (1% B to 99% B), 1 min at 99% B |

*Parameters may be adjusted to maintain system performance and analyte sensitivity.

Bromobimane (BB) and Bromobimane internal standard (B-IS) were injected before each calibration curve. B-IS was also injected after each highest calibration standard. Calibration curves were run before and after all clinical samples were run. QC's were injected every 50 samples. The analytical column was protected by a guard column or inline filter. Source settings were optimized for analyte sensitivity at the working flow rate to ensure efficient transmission of parent ions into the mass spectrometer. Detection of analytes was achieved using MRM settings in both positive and negative ionization modes. Statistical calculations and analytical run acceptance were determined based on criteria set forth in the FDA/EMA guidance where applicable. Fundamental parameters such as accuracy, precision, selectivity, sensitivity, reproducibility, and stability were monitored.

### Results

The data analysis was performed using the statistical analysis package JMP (Version 11.2, SAS). Correlation coefficients were determined for H₂S and each of the two SQOR products, thiosulfate and glutathione persulfide, using data from all subjects at all-time points. EPI-743 treated patients had a lower H₂S/thiosulfate ratio and a lower H₂S/glutathione persulfide ratio than untreated patients.

| | Correlation Coefficient | Correlation Coefficient |
|---|---|---|
| | (p-value) | (p-value) |
| SQOR-H₂S/Analyte | (−) EPI-743 | (+) EPI-743 |
| H₂S/Thiosulfate | 0.5946 | 0.4735 |
| | (<0.0001) | (<0.0001) |
| H₂S/Glutathione | 0.5024 | 0.3743 |
| persulfide | (<0.0001) | (0.0001) |

### EXAMPLE 3

### Thiosulfate Production By EPI-743 Treated Q7 Cells

Conditionally immortalized wild-type mouse striatal Q7 cells were seeded at a cell density of 50,000 cells/well of a 24-well plate in 0.5 mL of DMEM (High Glucose, 25 mM) medium containing 10% FBS, penicillin/streptomycin, and 400 μg/mL G418. The following day, the cells' medium was removed by aspiration and replaced with fresh medium containing either DMSO (0.3% v/v final concentration) or EPI-743 (1 μM final concentration). At 1 hour, 2.5 hours, 5 hours, and 24 hours post-dosing, 25 μL of media was transferred to cluster tubes containing thiol capping reagent. After briefly mixing, the tubes were frozen on dry ice and stored at <−70° C. until analysis.

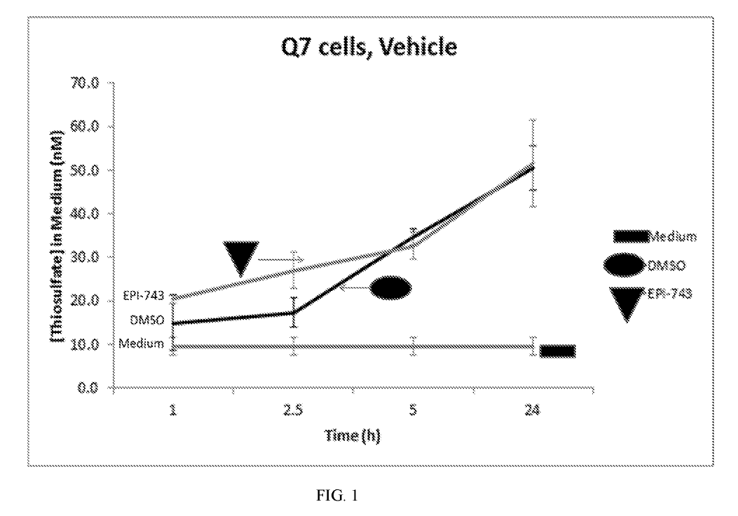

For thiosulfate analysis, samples were thawed in a room temperature water bath for 20 minutes. Media was precipitated with ice cold acetonitrile in a ratio of 1:1 (v:v). Following brief mixing, media samples were centrifuged at 4000 RPM for 15 minutes at 4° C. Supernatant was transferred to an injection plate and diluted with mobile phase A (water with 0.1% formic acid) in a 1:4 (v:v). Processed media samples were injected on a LC-MS/MS instrument for quantitation of Thiosulfate concentration at 1, 2.5, 5, and 24 hours after dosing. See FIG. 1.

### EXAMPLE 4

### Thiosulfate Production in EPI-743 Treated ATG-Stressed Cells

Conditionally immortalized wild-type mouse striatal cells (Q7, passage #45) were seeded at 300,000 cells per well in 6-well plates containing 3 mL/well of DMEM (High Glucose) containing 10% FBS, penicillin/streptomycin, and 400 μg/mL G418. After an overnight incubation at 33C, the cells were treated for 4-5 hours with either EPI-743 (100 nM final concentration) or DMSO vehicle (1% final concentration). Aurothioglucose (final concentration 10 μM) was added prior to an 18-hour treatment period. Cells were washed 1× with Hanks' buffered saline solution (HBSS), counted, and then resuspended in monobromobimane capping solution.

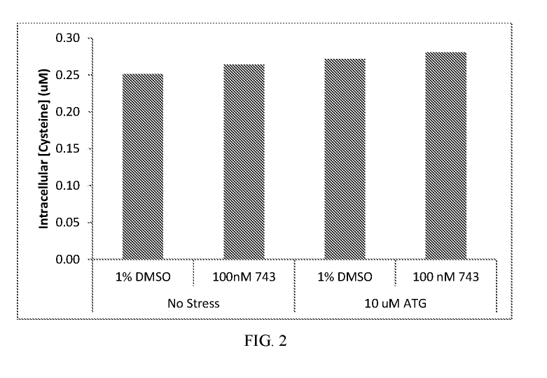

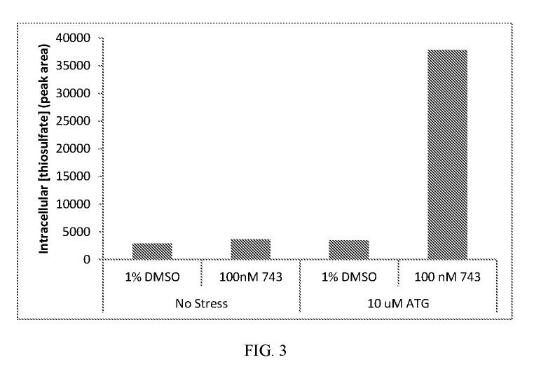

Cysteine and thiosulfate levels are shown below. Aurothioglucose (ATG) is an inhibitor of thioredoxin reductase-1 and has been repeatedly used as a tool compound to increase oxidative stress. The ATG-stressed cells showed an approximately 10-fold increase in thiosulfate in the EPI-743 treated versus untreated cells. Cysteine levels were unaffected by ATG or EPI-743 treatment. See Table 2 and FIGS. 2 and 3.

TABLE 2

| | | No Stress | | 10 μM ATG | |
|---|---|---|---|---|---|
| | | 1% | 100 nM | 1% | 100 nM |
| | | DMSO | 743 | DMSO | 743 |
| Calculated | Cysteine | 0.251 | 0.264 | 0.272 | 0.281 |
| Concentra- | | | | | |
| tion (μM) | | | | | |
| Peak Area | Thiosul- | 2881 | 3656 | 3466 | 37903 |
| | fate | | | | |

### EXAMPLE 5

### SQOR Expression and Purification

Human sulfide:quinone oxidoreductase (SQOR) protein was expressed and purified according to the method described in Jackson, M.R., et al. “Human Sulfide:Quinone Oxidoreductase Catalyzes the First Step in Hydrogen Sulfide Metabolism and Produces a Sulfane Sulfur Metabolite”, Biochemistry, Publication Date (Web): 1 Aug 2012, with the following exceptions: (1) a chaperone plasmid set (Plasmid pG-KJE8, plasmid #1 in catalog no. 3340) from Takara Bio Inc. (Japan) was used; (2) DHPC detergent was replaced with the following: 1.0% (w/v) CHAPS, 0.5% deoxycholate, or 1.0% octylmaltoside. The detergent was used in the initial lysis buffer, and removed in the IMAC purification step (no detergents in the storage conditions); (3) the ion-exchange (Q-sepharose) chromatography step was not used in the purification. The final SQOR protein contained amino acids 42-450 of the human SQOR protein.

Target protein was purified on IMAC to ≥90% purity (on Ni resin). Target protein was stored in 25 mM Tris pH 7.5, 150mM NaCl, 2 mM DTT 20% glycerol.

### EXAMPLE 6

### Determination of Kinetic Parameters of EPI-743 with SQOR

EPI-743 was assayed for activity with SQOR. This redox active compound may interconvert between the reduced (hydroquinone) and oxidized (quinone) forms, in particular the possible oxidation of hydroquinone to quinone by oxygen in solution. Accordingly, to limit non-enzymatic conversion of hydroquinone to quinone and avoid subsequent complications for the enzymatic activity analysis, the assays were performed under reduced oxygen atmosphere (<1000 ppm) in a controlled-atmosphere glovebox.

Briefly, SQOR enzyme, compound, inorganic sulfide (200 micromolar Na₂S) and a sulfur acceptor (2 mM Na₂SO₃) were brought together. The reduction of the quinone substrate to hydroquinone product in the presence of sulfide ion, sulfur acceptor and SQOR enzyme was monitored by the decrease in quinone concentration over time, by observing the UV signal diagnostic for the specific quinone (See Table 3):

TABLE 3

| Compound | | Δε |
|---|---|---|
| No. | Wavelength | (Ox-red)* |
| EPI-743 | 266 nm | 7450 M−1 cm−1 |

*Δε (Ox-red) at the listed X values in order to calculate rates of change in concentration of the quinones

For the above compound, the absorbance wavelength utilized was also the λₘₐₓ for that compound.

General assay conditions: Assays were performed in 100 mM Tris, pH 7.5, 0.5 mM EDTA. SQOR was included at approximately 5 nM, as estimated by total protein concentration determined by Bradford assay with BSA as standard and the estimated purity of SQOR from SDS-PAGE. Quinone substrates were added to concentrations between approximately 300 nM and 250 mM from 100× DMSO stocks, leading to 1% residual DMSO in assays. For compounds with difficult solubility, reduced Triton X-100 (Sigma X100 RS, CAS: 92046-34-9) was included at approximately 0.1% (v/v). This detergent was chosen as Triton X-100 and similar detergents contain a phenyl moiety which interferes with the UV monitoring described. Temperatures were maintained between 25 and 30° C. Assays were performed in either screwcap cuvettes purged with nitrogen or in plate format in a controlled atmosphere chamber in volumes from approximately 50-1000 μL, adjusting the pathlengths accordingly.

The kinetic parameters of compounds with SQOR were determined by varying the concentration of the compound over a suitable range and interrogating the impact of that variation on the enzymatic rate.

The parameters k_{cat} and Kₘ, for example, can be determined by nonlinear fitting of the rate data to the Michaelis-Menten equation:

Rate=k_{cat}*[S]/(Kₘ+[S])

or a derivative, where [S] is the concentration of the substrate (here, the compound being tested). These kinetic parameters individually and combined into the “specificity constant” (a second-order rate constant) k_{cat}/Kₘ provide information regarding the interactions between the substrate and enzyme. In particular, relatively large values of k_{cat}/Kₘ (exceeding ˜10⁴M⁻¹s⁻¹), suggest compounds with potential to significantly influence SQOR activity.

The following table shows kinetic parameters for the compound tested.

Kinetic parameters.

| Compound | k_{cat} | Kₘ | k_{cat}/Kₘ |
|---|---|---|---|
| No. | (s⁻¹) | (micromolar) | (s⁻¹M⁻¹) |
| EPI-743 | 76 | 200 | 0.4 × 10⁶ |

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is calimed is:

1. A method of increasing a thiosulfate level, decreasing a hydrogen sulfide level, or preventing an increased hydrogen sulfide level in a subject, comprising: administering to the subject an effective amount of an agent selected from the group consisting of:
compounds of Formula A, Formula I, Formula I-Unsat, Formula I-Sat, Formula II, Formula II-Unsat, Formula II-Sat, Formula III, Formula III-Unsat, Formula III-Sat, Formula IV, Formula IV-Unsat, Formula IV-Unsat-R, Formula IV-Unsat-S, Formula IV-Sat, Formula IV-Sat-R, Formula IV-Sat-S, Formula V, Formula V-Unsat, Formula V-Sat, Formula VI, Formula VI-Unsat, Formula VI-Unsat-R, Formula VI-Unsat-S, Formula VI-Sat, Formula VI-Sat-R, Formula VI-Sat-S, a hydroquinone of Formula I, a hydroquinone of Formula I-Unsat, a hydroquinone of Formula I-Sat, a hydroquinone of Formula II, a hydroquinone of Formula II-Unsat, a hydroquinone of Formula II-Sat, a hydroquinone of Formula III, a hydroquinone of Formula III-Unsat, a hydroquinone of Formula III-Sat, a hydroquinone of Formula IV, a hydroquinone of Formula IV-Unsat, a hydroquinone of Formula IV-Unsat-R, a hydroquinone of Formula IV-Unsat-S, a hydroquinone of Formula IV-Sat, a hydroquinone of Formula IV-Sat-R, a hydroquinone of Formula IV-Sat-S, a hydroquinone of Formula V, a hydroquinone of Formula V-Unsat, a hydroquinone of Formula V-Sat, a hydroquinone of Formula VI, a hydroquinone of Formula VI-Unsat, a hydroquinone of Formula VI-Unsat-R, a hydroquinone of Formula VI-Unsat-S, a hydroquinone of Formula VI-Sat, a hydroquinone of Formula VI-Sat-R, a hydroquinone of Formula VI-Sat-S, alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, alpha-tocopherol quinone, beta-tocopherol quinone, gamma-tocopherol quinone, delta-tocopherol quinone, alpha-tocopherol hydroquinone, beta-tocopherol hydroquinone, gamma-tocopherol hydroquinone, delta-tocopherol hydroquinone, and salts, stereoisomers, mixtures of stereoisomers, hydrates, and solvates thereof.

2.-64. (canceled)
